(19) United States
(12) Patent Application Publication
(10) Pub. No.: US 2023/0285617 A1
(43) Pub. Date: Sep. 14, 2023

(54) STERILIZATION DEVICE FOR SANITARY EQUIPMENT

(71) Applicant: OXTI CORPORATION, New Taipei City (TW)

(72) Inventor: Chih-Feng Ho, New Taipei City (TW)

(21) Appl. No.: 17/688,920

(22) Filed: Mar. 8, 2022

(51) Int. Cl. A61L 2/22; A61L 2/10; A61L 2/26; E03D 9/00

(52) U.S. Cl. CPC A61L 2/22; A61L 2/10; A61L 2/26; E03D 9/00; A61L 2101/34

(57) ABSTRACT

A sterilization device for sanitary equipment, applicable to a toilet, the sterilization device including: a controller; a disinfectant sprayer, provided with a storage container and sprayer, the disinfectant sprayer in electric connection with the controller; and a disinfection lamp, in electric connection with the controller. The present invention controls the disinfectant sprayer or disinfection lamp through the controller to disinfect the sanitary equipment of the toilet, for example, to disinfect stool, bathtub or sink by spraying alcohol or emitting ultraviolet light. Furthermore, the controller may be in connection with the timer; when a predetermined time set by the timer has expired, the controller controls the opening or closing of the disinfectant sprayer, or the on and off of the disinfection lamp, thereby improving use safety and convenience.

### TECHNICAL FIELD OF THE INVENTION

The present invention relates to a sterilization device, and more particularly to a sterilization device for sanitary equipment.

### DESCRIPTION OF THE PRIOR ART

Chinese Patent No. CN108374461B, titled “Induction flushing device for sanitary ware”, as FIG. 1 of the patent shows, structurally includes a toilet seat, a toilet lid, a flushing device, and a barrel body.

However, the above patent does not provide disinfection and sterilization functions. Sanitary equipment such as stool, bathtub and sink commonly used in family life stay wet for long periods of time during use, which is very easy to breed bacteria, molds, viruses and other pathogens, and influences human health. In this regard, it is very necessary to disinfect and sterilize sanitary products, so the conventional technology still has room for improvement.

### SUMMARY OF THE INVENTION

To achieve the above object, the present invention provides a sterilization device for sanitary equipment, applicable to a toilet, the sterilization device including: a controller; a disinfectant sprayer, provided with a storage container and sprayer, the disinfectant sprayer in electric connection with the controller; and a disinfection lamp, in electric connection with the controller,
where the controller is in connection with a timer,
where the sterilization device further comprising a fan in electric connection with the controller,
where disinfectant is filled inside the storage container of the disinfectant sprayer, and the disinfectant is alcohol.
where the disinfection lamp is a ultraviolet (UV) lamp or short-wave ultraviolet (UVC) lamp.

The present invention controls the disinfectant sprayer or disinfection lamp through the controller to disinfect the sanitary equipment of the toilet, for example, to disinfect stool, bathtub or sink by spraying alcohol or emitting ultraviolet light. Furthermore, the controller may be in connection with the timer; when a predetermined time set by the timer has expired, the controller controls the opening or closing of the disinfectant sprayer, or the on and off of the disinfection lamp, thereby improving use safety and convenience.

### BRIEF DESCRIPTION OF THE DRAWINGS

FIG. 1 is a structural block diagram of a sterilization device for sanitary equipment of the present invention;

FIG. 2 is a structural block diagram of the sterilization device for sanitary equipment of the present invention provided with a timer;

FIG. 3 is a schematically perspective view of the sterilization device of the present invention configured on a stool;

FIG. 4 is a schematically perspective view of the sterilization device of the present invention configured on a bathtub; and

FIG. 5 is a schematically perspective view of the sterilization device of the present invention configured on a sink.

### DETAILED DESCRIPTION OF THE PREFERRED EMBODIMENTS

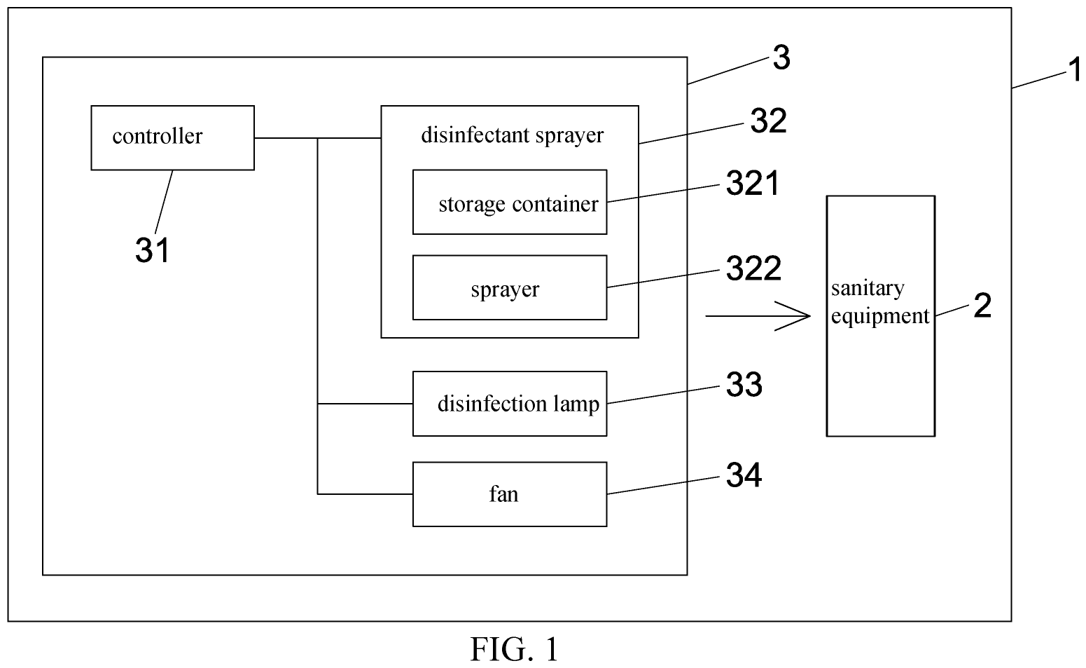

Referring to FIG. 1, the present invention provides a sterilization device 3 for sanitary equipment, applicable to a toilet 1, to disinfect and sterilize a sanitary equipment 2 of the toilet 1, where the sterilization device 3 includes a controller 31, disinfectant sprayer 32, and disinfection lamp 33.

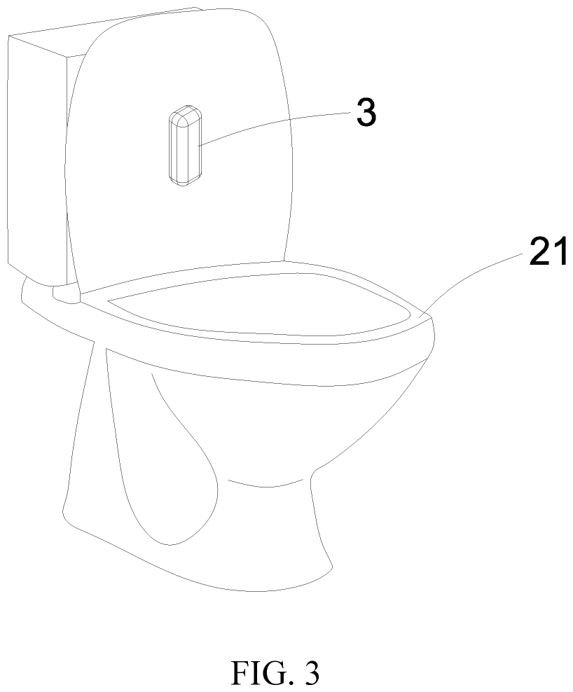

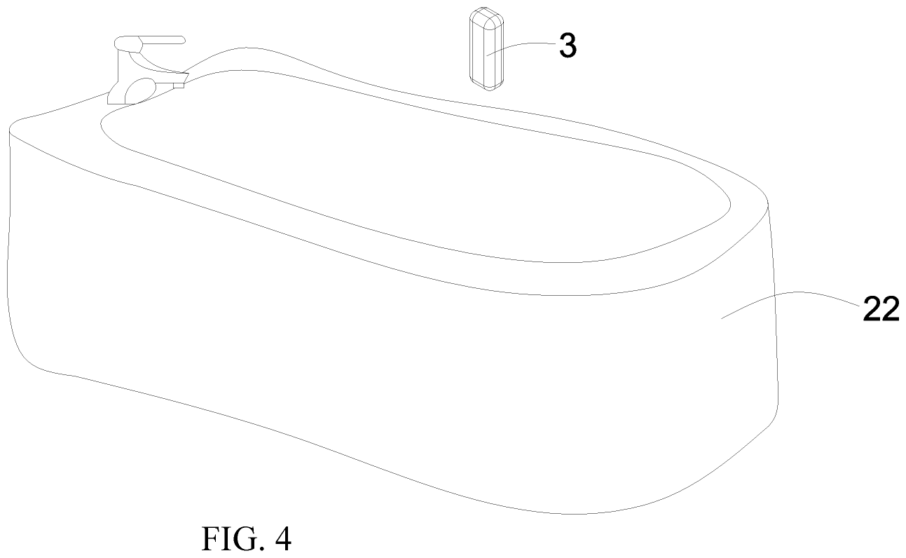

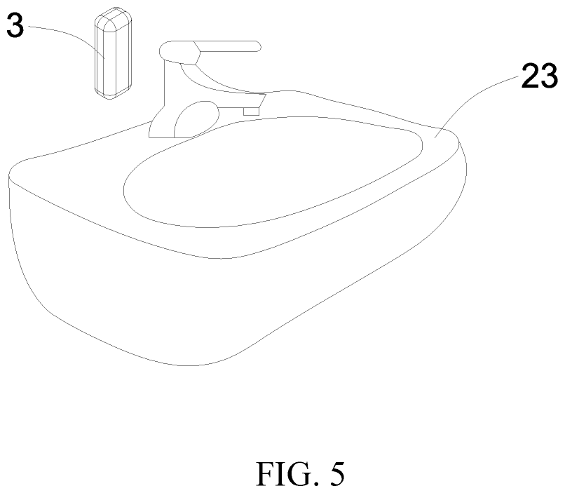

In the present invention, the toilet 1 is the collective name of a toilet, restroom and bath. The sanitary equipment 2 mainly refers to a stool 21 (marked in FIG. 3), a bathtub 22 (marked in FIG. 4), and a sink 23 (marked in FIG. 5).

The controller 31 may be a central processing unit (CPU), single-chip microcomputer or microcontroller (MCU).

The disinfectant sprayer 32 is provided with a storage container 321 and sprayer 322, where the disinfectant sprayer 32 and controller 31 are in electric connection with each other. Specifically, the inside of the storage container 321 of the disinfectant sprayer 32 is filled with disinfectant, which may be alcohol, but the present invention is not so limited, and it may be other disinfectant such as chlorine dioxide or sodium hypochlorite. The sprayer 322 is used to extract the disinfectant in the storage container 321 and spray the disinfectant.

The disinfection lamp 33 is in electric connection with the controller 31. Specifically, the disinfection lamp 33 may be a ultraviolet (UV) lamp. Furthermore, the ultraviolet lamp my be a short-wave ultraviolet (Ultraviolet-C, UVC) lamp according to the difference of the ultraviolet wavelength of the ultraviolet lamp, but the present invention is not so limited. The disinfection lamp 33 can emit ultraviolet light.

The sterilization device 3 may further include a fan 34 in electric connection with the controller 31.

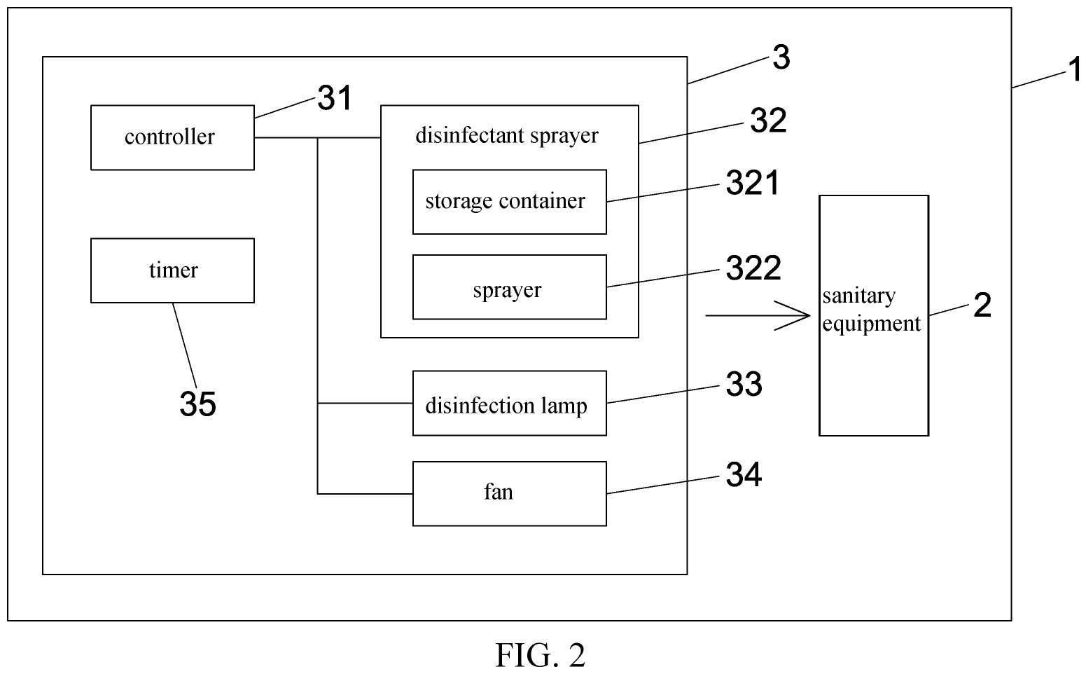

Referring to FIG. 2, In addition to the sterilization device 3 including all the components described in FIG. 1, the controller 31 can also be connected to a timer 35, which is used to provide a timing function; when a predetermined time set by the timer 35 has expired, the opening or closing of the disinfectant sprayer 32, or the on or off of the disinfection lamp 33 is controlled through the controller 31.

For example, when performing sterilization, disinfection and sterilization can be specified at the predetermined time (e.g., 12:00 in the middle of the night). Upon the disinfection and sterilization, the controller 31 controls the disinfectant sprayer 32 to open and spray the disinfectant to the sanitary equipment 2, or the disinfection lamp 33 to turn on and emit ultraviolet light to the sanitary equipment 2. Referring to FIGS. 3 to 5, the disinfection and sterilization are respectively performed on the stool 21 (marked in FIG. 3), bathtub 22 (marked in FIG. 4), and sink 23 (marked in FIG. 5).

For another example, when performing sterilization, the controller 31, in a sterilization state, controls the disinfectant sprayer 32 to spray disinfectant to the sanitary equipment 2, and controls the disinfectant sprayer to close again after the timer 35 counts for a few seconds, or the controller 31 controls the disinfection lamp 33 to emit ultraviolet light on the sanitary equipment 2, and controls the controller 31 to turn off again after the timer 35 counts for a few seconds.

The controllers 31 can also control the fan 34 to supply air to the sanitary equipment 2, so as to dry the disinfectant or water vapor on the sanitary equipment 2, preventing excessive humidity.

To sum up, the present invention controls the disinfectant sprayer 32 or disinfection lamp 33 through the controller 31 to disinfect the sanitary equipment 2 of the toilet 2, for example, to disinfect stool, bathtub or sink by spraying alcohol or emitting ultraviolet light. Furthermore, the controller 31 may be in connection with the timer 35; when a predetermined time set by the timer 35 has expired, the controller 31 controls the opening or closing of the disinfectant sprayer 32, or the on and off of the disinfection lamp 33, thereby improving use safety and convenience.

I claim:

1. A sterilization device for sanitary equipment, applicable to a toilet,
said sterilization device comprising:
a controller;
a disinfectant sprayer, provided with a storage container and sprayer,
said disinfectant sprayer in electric connection with said controller; and
a disinfection lamp, in electric connection with said controller.

2. The device according to claim 1, wherein said controller is in connection with a timer.

3. The device according to claim 1, further comprising a fan in electric connection with said controller.

4. The device according to claim 1, wherein disinfectant is filled inside said storage container of said disinfectant sprayer, and said disinfectant is alcohol.

5. The device according to claim 1, wherein said disinfection lamp is a ultraviolet (UV) lamp or short-wave ultraviolet (UVC) lamp.
